(19) United States
(12) Patent Application Publication
(10) Pub. No.: US 2017/0027454 A1
(43) Pub. Date: Feb. 2, 2017

(54) Capillary Refill Measurement

(71) Applicant: The University of Nottingham, Nottingham, Nottinghamshire (GB)

(72) Inventors: Sergiy Korposh, Nottingham (GB); Stephen Morgan, Nottingham (GB); Barrie Hayes-Gill, Nottingham (GB)

(21) Appl. No.: 15/303,189

(22) PCT Filed: Apr. 8, 2015
(86) PCT No.: PCT/GB2015/051071

§ 371 Date: Oct. 10, 2016

(30) Foreign Application Priority Data
Apr. 8, 2014 (GB) 1406343.2

(51) Int. Cl. A61B 5/02; G01G 19/50; A61B 5/026; A61B 5/103; A61B 5/00; A61B 5/0205

(52) U.S. Cl. CPC A61B 5/02028; A61B 5/6807; A61B 5/6892; A61B 5/02055; A61B 5/0261; A61B 5/1036; A61B 5/0022; G01G 19/50; A61B 2562/0233

(57) ABSTRACT

A capillary refill measurement apparatus (1), comprising a support element (14) configured to receive the weight of a user through their foot, a light source (22) for illuminating a region of the foot that exerts weight onto the support element (14), a light detector (24) arranged to receive light from the illuminated region (36) of the foot and generate an output based on the received light, and a processor (26) configured to determine a capillary refill rate from the output of the light detector (24) when the user adjusts the amount of weight received by the support element (14).

The present invention relates to a method and apparatus for measuring capillary refill time. More specifically, the invention relates to measuring capillary refill time at a foot.

Individuals with diabetes and vascular disease have an increased risk of developing foot ulcers and complications. Such complications can be severe and ultimately may result in amputation. Disease of the foot in diabetes represents a major health risk and a source of very considerable cost and suffering. Over 80% of people who lose a leg in diabetes initially present with a foot ulcer (Pecoraro et al. Diabetes Care 13:513-521, 1990). Major contributory risk factors for the development of foot ulcers and tissue breakdown are impaired peripheral bloodflow and peripheral neuropathy with the loss of ability to feel pain, temperature or pressure sensation in the feet and lower legs (Boulton et al. www.thelancet.com 366, 2005).

Foot ulceration affects approximately 15% of people with diabetes and can be difficult to treat: only two thirds heal in six months and the cost to the NHS in the UK is 2010-2011 was £650-750M (Kerr, Foot Care For People With Diabetes, www.diabetes.nhs.uk, 2011). The burden of care in relation to foot ulcers and related foot injuries is increasing with the ever increasing prevalence of Type 2 diabetes (Vamos et al, Diabetes care 33:2592-2597, 2010). It has been recently reported that approximately 100 amputations are performed each week in the UK, 85% of which are deemed to be avoidable (Holman et al. Diabetologia 2012).

J. Spigulis et al (Wearable wireless photoplethysmography sensors, Biophotonics: Photonic Solutions for Better Health Care, Proc SPIE Vol. 6991, doi 10.1117/12.801966) discloses a photoplethysmography (PPG) sensor that may be integrated within a sock, hat or glove. The pulse wave transit time, determined by the time delay between a pulse at the hat and the glove, is used to infer blood pressure. The PPG sensor is not used to monitor circulation in the foot.

A method and apparatus that can provide prophylactic treatment of the type of foot injuries that may result from diabetes is desired, to reduce the cost and suffering associated with such injuries.

According to a first aspect of the invention, there is provided a capillary refill measurement apparatus, comprising a light source for illuminating a region of a foot of a user, a light detector arranged to receive light from the illuminated region of the foot and generate an output based on the received light, a processor and a support element. The support element is configured to receive the weight of a user acting through the foot. The processor is configured to determine a capillary refill rate from the output of the light detector when the user adjusts the amount of weight received by the support element. The illuminated region of the foot may be adjacent to the support element. The illuminated region of the foot may be a region of the foot that exerts weight onto the support platform when the support platform receives the weight of the user.

Such an apparatus enables monitoring of the circulation of the user's foot, for example whilst the user is walking around, so that any reduction in circulation may be detected early, and appropriate actions taken to prevent injury to the user's foot. The provision of a device that is for standing on enables rapid measurement of circulation as the user shifts their weight, in contrast to approaches in which an object is pressed into the user's skin using an externally applied force whilst the user is static. An apparatus that can support a user's weight at an illuminated region substantially simplifies determination of capillary refill at the user's feet, and monitoring it whilst the user moves around, for example, in daily use, may be better as a useful measurement in some circumstances.

The apparatus may further comprise a force or pressure transducer, configured to provide an output based on the amount of weight supported by the support element. The pressure transducer may allow the changes in light detected at the illuminated region to be corrected for the magnitude and/or duration of the pressure applied prior to capillary refill, thereby enhancing the accuracy of the measurement.

The pressure transducer may comprise an electrically conductive woven fabric that changes impedance (e.g. changes resistance) in response to pressure. The use of an electrically conductive woven fabric simplifies the integration of a pressure sensor with a wearable item, enabling a low profile pressure sensor that is comfortable and unobtrusive.

The apparatus may further comprise a temperature sensor configured to provide an output related to the temperature of the foot, when the apparatus is in use. Directly measuring the temperature of the foot near to the illuminated region (for example within 2 cm) may provide optimal data for temperature correction of the capillary refill time.

When determining a capillary refill rate the processor may be responsive to at least one of a pressure on the support element and the temperature of the foot. Use of the temperature and pressure readings may enhance accuracy and consistency, enabling relevant comparisons to be made between capillary refill times measured under different conditions. The processor may be remote from the foot of the user.

The apparatus may comprise an illumination optical fibre for directing light from the light source to the illuminated region and a sensing optical fibre for directing light from the illuminated region to the light detector. Optical fibres enable a light source that is remote from the illuminated region to be used, improving the range of target applications for the device. Optical fibres can be integrated with a wearable item without compromising comfort because they are relatively thin and flexible.

The illumination optical fibre and/or the sensing optical fibre may comprise a stripped portion in which light is transmissible through a sidewall of the optical fibre.

A tip of the illumination optical fibre that is adjacent to the illuminated region may be at an angle of between 25° and 180°, or between 25° and 155° to a direction of a tip region of the sensing optical fibre that is adjacent to the illuminated region. The tip region of the illumination optical fibre may be at an angle of between 75° and 105° to the tip of the sensing optical fibre. The applicant has found that these angular ranges to reduce the change in detected light as a result of movement of the user. In these angular ranges, the fibres are said to be at 0° when they are end to end, and their axes are co-incident. When the fibres are side by side, parallel and facing in opposite directions, they are at an angle of 180°. Avoiding an end to end arrangement may be advantageous, because this arrangement may increase movement artefacts.

The illumination and/or sensing optical fibre may comprise or consist of a polymer optical fibre. Plastic optical fibres may be relatively low cost, with sufficient performance. A cladding of the optical fibre may comprise a fluorinated polymer. A core of the optical fibre may comprise PMMA (Poly(methyl methacrylate)) or a perflourinated polymer (e.g. polyperfluorobutenylvinylether).

The illumination and sensing optical fibre may comprise a cladding and a core. A stripped portion may be a region from which cladding has been at least partially removed, such that light within the illumination fibre will more readily pass through a sidewall of the core. The surface of the core in the stripped portion may be roughened, in order to enhance the transmission of light through the sidewall of the core in the stripped portion.

The stripped portion may be formed by abrading the cladding of the illumination and/or sensing optical fibre. The core may also be at least partially abraded (for example to enhance surface roughness thereof).

A stripped portion of the illumination optical fibre may be arranged adjacent to a stripped portion of the sensing optical fibre. Light may thereby be coupled from the illuminating optical fibre to the sensing optical fibre via an adjacent foot.

The illumination and sensing optical fibre may be substantially parallel in the region of the stripped portions thereof.

The apparatus may comprise a sensing region, comprising an illumination optical fibre and a first and second sensing optical fibre arranged on either side of the illumination optical fibre. The sensing region may comprise stripped portions of the illumination optical fibre and stripped portions of the first and second sensing optical fibre.

The stripped portions of the illumination optical fibre and first and second optical fibre may be substantially parallel.

A length of the stripped portion may be 1 mm to 15 mm. The length of the stripped portion may be 5 mm to 10 mm.

A spacing between adjacent stripped portions may be 0.5 mm to 2 mm. The spacing may be around 1 mm.

The support element may comprise a fabric, such as a woven fabric. The use of fabric, simplifies the integration of a pressure sensor with a wearable item. At least one of the illumination and sensing optical fibre may be woven into the fabric of the support element. This minimises the impact of the illumination and sensing means on the comfort of the user.

The sensing optical fibre (or fibres) and the illumination optical fibre (or fibres) may be knitted in the fabric such that these optical fibres are substantially parallel.

The apparatus may comprise a wireless transmitter for transmitting information from the processor to a remote computer. Wirelessly transmitting data allows alerts to be sent to the user (or their healthcare professional) based on the data, without the need for connecting the apparatus to an external computer via a wired connection. For instance, the remote computer may determine that the capillary refill rate indicates a health problem, and may send a notification to the user to alert them of this. An application may be provided for a smartphone for receiving data from the apparatus, which is operable to generate an alert based on a predetermined threshold. The application may be configured to automatically forward notifications to third parties (such as a nurse or doctor) based on the capillary refill rate.

The apparatus may comprise a housing in which the light source, light detector and processor are disposed.

The housing may comprise a connector for removably coupling at least one of: the illumination and optical fibre to the light source and the sensing optical fibre to the light detector. Such an arrangement allows the housing to be re-used. The item comprising the support element may be disposable, and the housing re-usable, thereby minimising the cost of using the apparatus. The item comprising the support element may be tailored for a particular user's needs, for example being designed to fit a user's foot. The housing may be generic, and connectable to a tailored item comprising the support element.

The housing may comprise a strap for mounting on an ankle of the user. This is a convenient way of supporting the housing in proximity to the foot.

The apparatus may be wearable by the user. A wearable device enables monitoring of circulation in the foot as the user is walking, and removes the need for a user to have to operate equipment that they do not already have with them. A wearable device can intermittently or regularly monitor health of circulation in a user's foot (for example at least once per day), and alert the user to any changes. For example, the user could be advised to take a break from walking. Alternatively, periods of inactivity in which no walking takes place could result in a prompt for the user to exercise in order to improve their general health.

The support element may comprise part of a sock. The support element may comprise part of an insole and/or part of a shoe.

According to a second aspect of the invention, there is provided a weighing scale comprising the apparatus according to the first aspect. The scale comprises a platform for a user to stand on, and may include a display for displaying the weight of the user on the platform. The platform comprises the support element and the light source and the light detector are attached to the platform. Standing on scales may be part of a regular routine for many people and checking of circulation in their feet can be integrated into such a routine if apparatus capable of performing such a measurement is integrated with their scales. Scales typically include a display suitable for indicating the weight of the user, which may also be used to indicate the capillary refill time and/or to make recommendations for the user to follow to improve or maintain the health of their feet.

According to a third aspect of the invention, there is provided a method of determining a capillary refill rate comprising illuminating a region of a foot of a user, measuring light from the illuminated region as the user shifts their weight on the illuminated region, and determining a capillary refill time from the measured light.

The method may be performed using the apparatus according to the first or second aspect. The method may comprise comparing the capillary refill rate with a normal value of a capillary refill rate. The normal value of capillary refill rate may be taken from an earlier time, or at a different spatial position, or on the opposite foot. The method may comprise sending an alert if the capillary refill rate deviates from the normal value. The method may comprise generating a recommendation to a user to change their behaviour based on the capillary refill time. The recommendation may be automatically generated. The recommendation may be communicated to a user's mobile phone or personal computer by the apparatus.

According to a fourth aspect of the invention, there is provided a capillary refill measurement apparatus, comprising a support element configured to receive the weight of a user through their foot, a light source for illuminating a region of the foot that exerts weight onto the support element, a light detector arranged to receive light from the illuminated region of the foot and generate an output based on the received light, wherein the output is suitable for performing a capillary refill measurement.

Optional features of the first and second aspect, and of the example embodiments described hereinafter, may be combined with the fourth aspect of the invention.

Embodiments of the invention will now be described, purely by way of example, with reference to the accompanying drawings, in which:

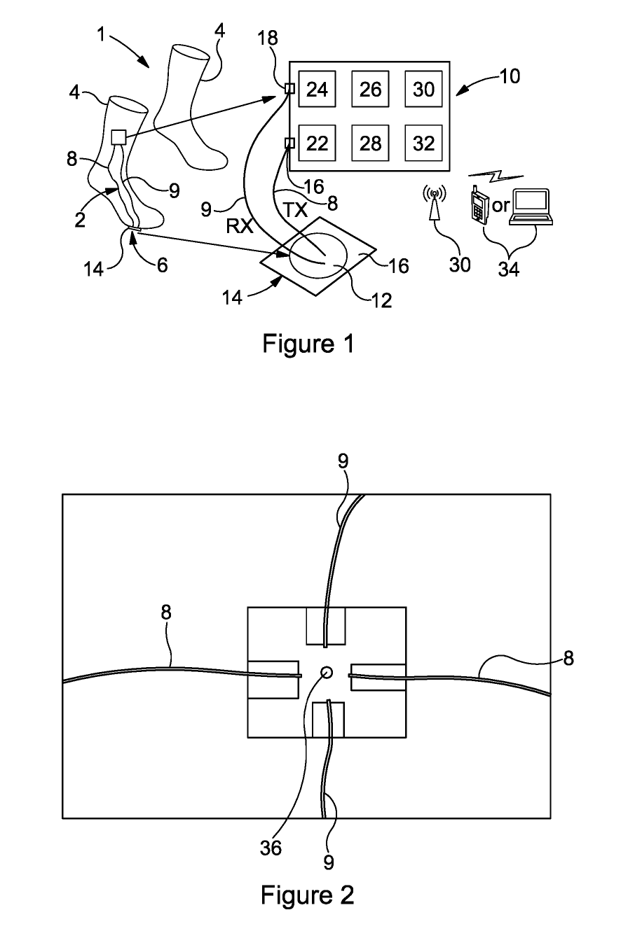

FIG. 1 is a schematic diagram of an apparatus according to an embodiment of the first aspect of the invention;

FIG. 2 is a schematic of an example arrangement of sensing an illumination optical fibres;

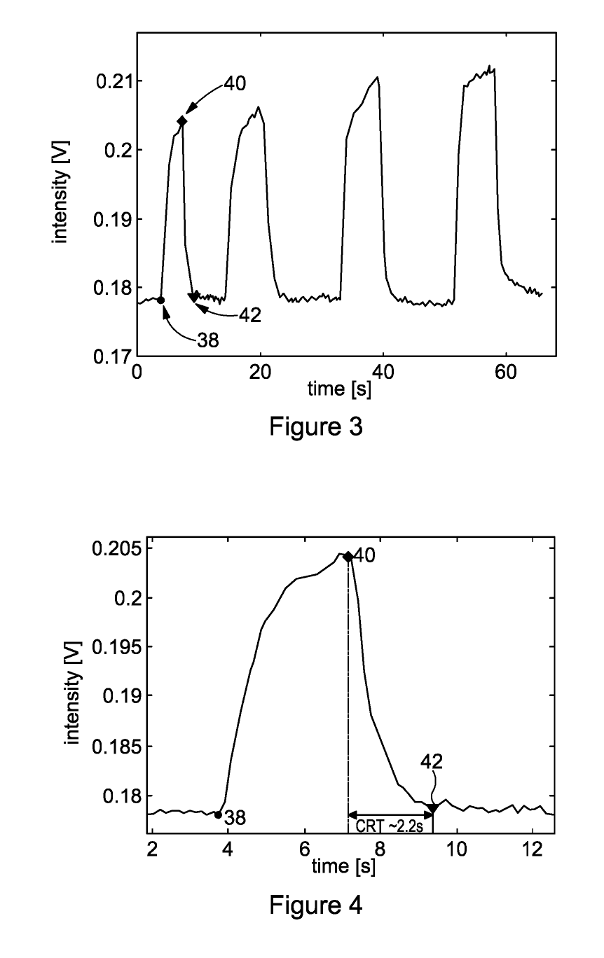

FIG. 3 is a graph showing light intensity measured under a foot by apparatus according to an embodiment of the invention;

FIG. 4 is a graph showing a single weight on, weight off, capillary refill cycle, measured under a foot by apparatus according to an embodiment of the invention;

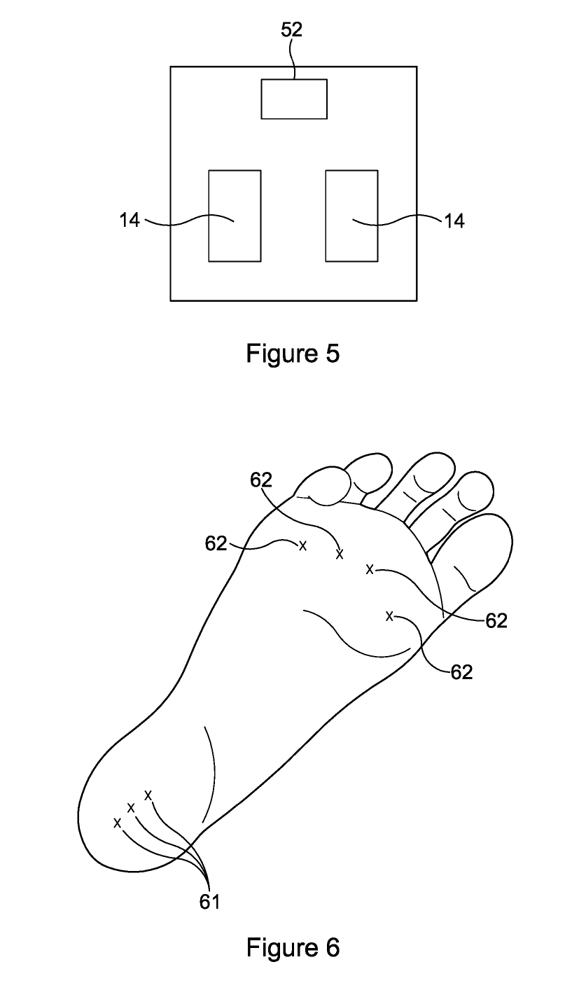

FIG. 5 is a schematic diagram of an embodiment of the second aspect of the invention;

FIG. 6 is a schematic of a foot showing example illuminated regions;

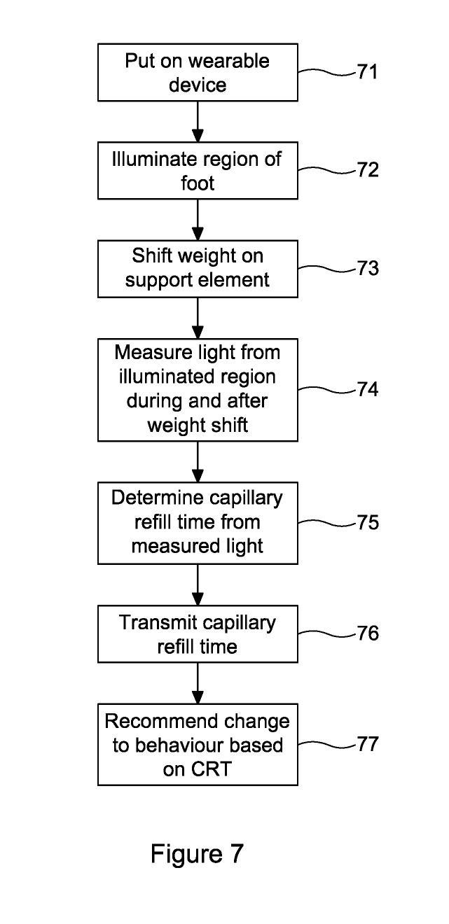

FIG. 7 is a flow diagram of a method of using the apparatus according to an embodiment;

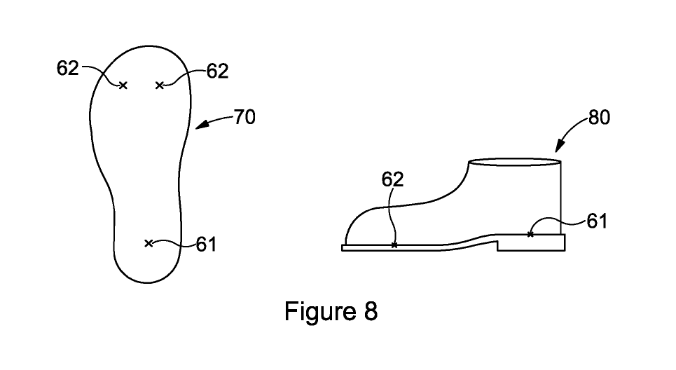

FIG. 8 is a diagram of embodiments integrated with an insole and with a shoe.

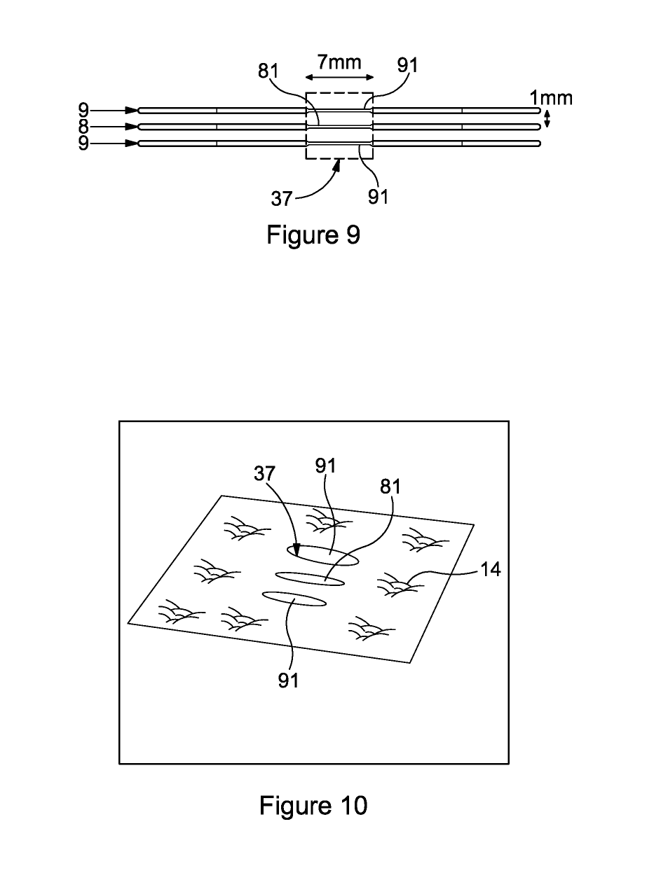

FIG. 9 is a schematic diagram of a sensing region defined by stripped portions of adjacent optical fibres;

FIG. 10 is a photograph of a sensing region defined by stripped portions of adjacent optical fibres;

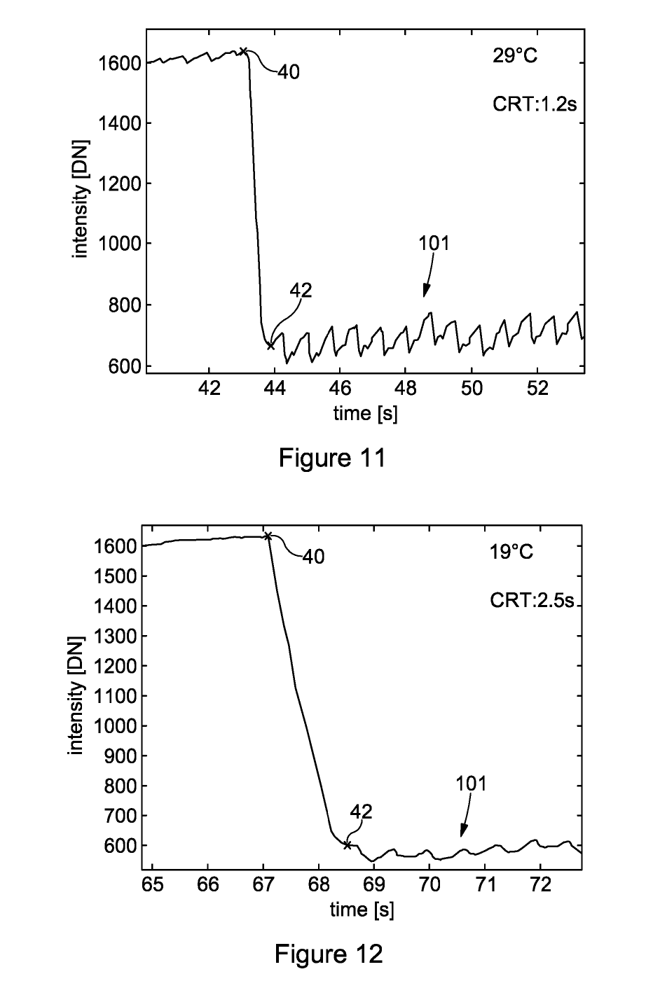

FIG. 11 is a graph of a capillary refill time for a warm foot measured using an apparatus according to an embodiment; and

FIG. 12 is a graph of a capillary refill time for a cold foot measured using an apparatus according to an embodiment.

Referring to FIG. 1, a pair of socks 4 are shown. The right sock 4 comprises apparatus 1 according to an embodiment of the invention. The apparatus 1 comprises a housing 10, support element 14 and a pair of optical fibres 2. The pair of optical fibres 2 comprise an illumination optical fibre 8 and a sensing optical fibre 9.

In the housing 10 are disposed a light source 22, light detector 24, analog to digital converter 26, processor 28, power source 30 and wireless transmitter/transceiver 32. A light source connector 16 is provided for coupling a first end of the illumination optical fibre 8 to the light source 22. A light sensing connector 18 is provided for coupling a first end of the light sensing optical fibre 9 to the light detector 24.

In this embodiment, the light source 22 is a light emitting diode (LED), but any suitable light source may be used (for instance a red light emitting diode, or some other type of light source). The light detector 22 is preferably a photodiode, but may be any suitable light sensor that provides the appropriate sensitivity and signal to noise ratio.

The respective second ends of the illuminating and sensing fibres 8, 9 are positioned adjacent to the surface of the support element 14. The support element 14 is a portion of the sock 4 that, when the sock 4 is worn by a standing or walking user, bears weight from the user. The illuminating optical fibre 8 is arranged on the support element 14 so that light leaving the second end of the illuminating optical fibre 8 illuminates the region of the foot that is adjacent to the support element 14. The second end of the sensing optical fibre 9 is positioned to receive the light from the illuminated region of the foot, at least some of which will be absorbed by blood that is present in the capillaries near the skin. A different amount of blood in the capillaries will result in a different amount of light being received by the sensing fibre 9.

When the user changes the weight onto the support element 14 through the foot, for instance by shifting their weight, or by standing, blood may be squeezed from the capillaries, resulting in a change in the light received by the sensing fibre 9. The reduction in blood in the capillaries results in a higher light intensity received by the sensing fibre 9, which is detected by the light detector 24. When the user reduces the weight on the support element 14, blood will refill the capillaries adjacent to the illuminated region, and this will again change the light intensity received by the sensing fibre 9 and detected by the light detector 24.

The sensing and illuminating optical fibres 8, 9 are preferably integrated with the fabric of the sock 4, for example by stitching, knitting or weaving the fibres 8, 9 through the fabric of the sock 4. The integration of the optical fibres 8, 9 can take place during fabrication of the sock, or after. For example, the fibres 8, 9 can be stitched through the sock 4, or glued or taped to it.

In some embodiments there may be a plurality of illumination optical fibres 8 and/or a plurality of sensing optical fibres 9. In some embodiments the light source 22 may comprise a plurality of light emitting elements (such as LEDs). A plurality of illuminating optical fibres 9 may be coupled to a single light emitting element, or a light emitting element may be provided for each illuminating optical fibre 9. Similarly, more than one light detector 22 may be provided, each connected to a different sensing optical fibre 9.

The light source 22 may be controlled by the processor 28. The light source 22 may be modulated (e.g. under the control of the processor 28), and the output from the light detector 22 may be demodulated (for instance by the processor 28), which may improve signal to noise ratio.

The signal from the light detector 24 is preferably provided to the processor 28 after conversion to a digital signal by an analog to digital converter (ADC) 26. The processor 28 may include the ADC 26, or the ADC 26 may be a separate part.

The power supply 30 is preferably a battery (e.g. a rechargeable battery) but may be a capacitor or any other device suitable for providing stored electrical energy to the other components within the housing 10.

The processor 28 is configured to communicate wirelessly, via the wireless transmitter/transceiver 32 and antenna 30, which may be a Bluetooth® or Zigbee® device. The processor 28 may thereby interface with an external computer 34, for instance a personal computer, smartphone or tablet. The interface with the external computer 34 may be one way, enabling upload of data from the processor 28, or may be two way, allowing the external computer to control, program or set preferences on the processor 28.

The support element 14 comprises a pressure sensor 12, configured to provide an output related to the amount of weight that is applied to the support element 14 by the user. The support element 14 may further comprise a temperature sensor 16 that detects the temperature of the region of the foot in contact with the support element 14. Both the pressure sensor 12 and the temperature sensor 16 provide their outputs to the processor 28, via the ADC 26. In some embodiments the temperature sensor is integrated with the support element 14. Alternatively, the temperature sensor may merely be close to the support element, for example within 1 mm, 10 mm, 20 mm, 30 mm or 40 mm of the support element 14.

The pressure sensor 12 is preferably integrated with the fabric of the sock 4. For instance, the pressure sensor 12 may be formed from a type of fabric that changes resistance when a load is applied thereto, such as the fabrics developed by Footfalls & Heartbeats Ltd of Auckland, New Zealand. Alternatively, the temperature sensor 16 may be a platinum wire type resistance based temperature sensor 16, a thermocouple, or a thermistor, and/or may be constructed from a fabric.

Wires connecting the pressure sensor 12 and temperature sensor 16 to the processor 28 are provided (not shown). The wires are preferably integrated with the fabric of the sock, for instance by stitching, knitting or weaving the wires through the fabric of the sock. A biasing and/or readout circuit may be used to derive measurements from the pressure sensor 12 and temperature sensor 16 (not shown).

FIG. 2 shows an example arrangement of optical fibres 8, 9 at the support element 14 in more detail. In this embodiment the fibres 8, 9 are secured to the support element 14 using tape, rather than integrated with the support element 14 by weaving, knitting or stitching them in place. The fibres 8, 9 are secured to an outward facing external surface of the support element 14, with a hole being present in the support element 14 through which the skin is illuminated and light is received. In other embodiments the tips of the optical fibres 8, 9 that are adjacent to the support element 14 may be at an inward facing surface of the support element 14, so that they can illuminate and receive light without the need for a hole in the support element 14. In this example, the hole defines the region 36 of the foot to be illuminated. In an alternative arrangement, a portion of the sock 4 adjacent to the illuminated region 36 may be transparent, and the foot may be illuminated through the transparent portion.

There are a pair of illumination fibres 8, and a pair of sensing optical fibres 9. The applicant has found that it is advantageous to arrange the illumination and sensing optical fibres at an angle to each other, so as to reduce motion artefacts in the sensed light intensity. An angle of 90 degrees between the illumination and optical fibres 8, 9 has been used in this embodiment. The applicant has found this angle between the illumination and sensing optical fibres 8, 9 to be particularly advantageous for rejecting movement artefacts.

The respective tips of the pair of illumination fibres 8 adjacent to the support element 14 are substantially parallel, and shine light at the illuminated region 36 from opposite directions. The respective tips of the pair of sensing optical fibres 9 are likewise substantially parallel, and respectively detect light scattered from the illuminated region 36 in two opposite directions, these directions being at approximately 90 degrees to the tips of the illumination fibres 8.

The gap between the tips of the illumination fibres is approximately 3 mm, and the gap between the tips of the sensing fibres is approximately 3 mm. The ends of the sensing and illumination optical fibres are all substantially equidistant from the centre of the illuminated region 36, and are at a distance of approximately 1.5 mm therefrom. In other embodiments the end of the sensing optical fibre (or fibres) may be further away, or closer to, the illuminated region that the end (or ends) of the illuminating optical fibre (or fibres).

In this embodiment the light source 22 is a green light emitting diode, and the light detector 24 is a photodiode. The processor 28 may be a low power embedded microcontroller.

The housing 10 may be miniaturised, and attached to a strap for mounting on the ankle of a user. Alternatively, the housing 10 may be attached to the fabric of the sock 2. The appropriate choice may depend on the size and weight of the housing 10 and the associated control electronics. The housing may be less than 2 cm in maximum extent, and may have a bounding box of dimensions less than 2 cm×2 cm×8 mm. The weight of the housing may be less than 100 g, preferably less than 50 g.

In some embodiments the power source may be external to the housing 10. The power source may comprise a major element of the size and weight

In some embodiments the connections 16, 18 between the optical fibres 8, 9 and the housing may be re-usable so that the housing 10 may be re-used with a plurality of socks 2 having integrated sensors. The sock 4 may be a low cost item and/or may be tailored to a specific user. The housing 10 and associated electronics may cost more, and may be generic and re-usable between different users. The wearable item (e.g. sock 4) may be free from electronic components, so that it is washable (e.g. by machine). The housing 10 may be waterproof and/or shockproof, and may, for example encapsulate the electronics (including the processor 26) in a polymeric and/or elastomeric material.

FIGS. 9 and 10 show an alternative arrangement of optical fibres, in which an illumination fibre 8 and a first and second sensing fibre 9 are arranged substantially parallel to each other. The first and second sensing fibres 9 are positioned to either side of the illumination fibre 8.

A sensing region 37 is defined by adjacent stripped portions 81, 91 of the illumination fibre 8 and first and second sensing fibres 9. In this example, the stripped portions 81, 91 are approximately 7 mm long, and the spacing between stripped portion 81, 91 is approximately 1 mm. At the stripped portions 81, 91, part of the cladding of the optical fibres 8, 9 has been removed so that light can pass through a sidewall of the fibre 8, 9. Light can pass through the side of stripped portion 81 of central illumination fibre 8 to illuminate an adjacent illuminated region of a foot. Light from the illuminated region is received in the sensing fibres 9 through the stripped regions 91.

A sidewall of a core of the optical fibre 8, 9 at the stripped portions 81, 91 may be roughened, in order to enhance transmission of light through the sidewall.

The same or a similar arrangement of light source and light detector to that described above can be used with this embodiment. The light from the first and second sensing fibres can be combined at a single detector, or a light detector can be provided for each sensing fibre 9.

FIG. 10 shows a photograph of a fabric support element 14 having a sensing region 37 defined by stripped portions 81, 91 of three adjacent optical fibres. The stripped portions 81, 91 may conveniently be formed by abrading the cladding. This is particularly straightforward when the optical fibre is a polymer optical fibre. The stripped portions 81, 91 are at the surface of the fabric, so that the apparatus can readily be positioned to illuminate an adjacent region of skin if it is incorporated into a wearable item, such as a sock.

FIGS. 3 and 4 show traces obtained from an embodiment of the invention. The light intensity determined by the detector 24 is plotted against time as the user shifts the weight on their feet, for example by lifting and standing on a foot. At t=0, no load is being placed on the support element 14, and the light detector 24 provides a baseline reading corresponding with skin that is perfused with blood. At a later time 38, weight is placed on the support element 14 by the foot, and the blood is squeezed away from the surface capillaries by the pressure, resulting in an increase in intensity, up to a maximum 40, which may correspond with a maximum load condition on the support element 14.

The load on the support element 14, and the adjacent region of the foot, is subsequently removed, and the capillaries at the illuminated region 36 refill. An increase in light intensity is associated with unloading of the support element 14. The time it takes for the light intensity to return to the baseline perfused condition 42 from the loaded condition may be the capillary refill rate. The processor 28 is configured to determine the capillary refill rate from the output of the light detector 24. The processor may be responsive to the output of the pressure sensor 12 and temperature sensor 16 in determining the capillary refill rate. It is well known that the capillary refill rate may show a substantially linear temperature dependency, and the temperature of the illuminated region (or a region nearby) may thereby be used to provide temperature compensation (for example by means of a lookup table). The output of the pressure sensor 12 may be used to trigger the timing of the capillary refill measurement, and/or the capillary refill measurement may be corrected based on the magnitude and/or duration of loading prior to unloading.

FIGS. 11 and 12 show further example traces, obtained from an embodiment. Rather than using a foot, a finger was used in these examples to demonstrate the apparatus. In FIG. 11, a warm finger is used, with a skin temperature of 29° C. The light intensity at t=42 s corresponds with the finger pressing on a sensing region of the apparatus. The load is removed at the time indicated by 40, and the capillaries in the illuminated region adjacent to the sensing region refill. The light intensity returns to the baseline perfused condition at the time indicated by 42. The time difference between time 42 and time 40 is the capillary refill time, which is 1.2 seconds in this case. The pulse signal 101 is visible in the baseline perfused condition.

In FIG. 12, the finger is cooled before the test (for instance in a cold water bath). The skin temperature for the data of FIG. 12 is 19° C. This results in vasoconstriction, which increases the capillary refill time. In this graph, the capillary refill time is 2.5 s.

Variations in capillary refill time resulting from temperature changes can clearly be detected by apparatus according to an embodiment.

In some embodiments, the processor 26 will perform a curve fitting analysis (for example based on regression), in order to fit a mathematical model to the curve, This may be a more robust measurement than simply timing the delay until the measured light intensity returns to a baseline. The use of a method that fits a mathematical function to the data may allow the capillary refill rate to be determined without needing the detected light to return to the baseline level. A capillary refill rate may be determined based on a plurality of load/unloading cycles (for example by averaging, or by any other appropriate technique, such as a maximum likelihood method).

A plurality of regions 36, 61, 62 of the foot may be illuminated, either by a plurality of light sources or by a single light source that is directed to each region (e.g. by optical fibres. There may be at least 3 illuminated regions, or at least 5 or 10 illuminated regions. The different regions may be positioned in different parts of the foot. For instance, where there are three illuminated regions, a first region may be on the heel, a second region may be near the ball of the foot (the first metatarsophalangeal joint), and a third region may be near a further metatarsophalangeal joint, for example the third such joint. In some embodiments there may be an array of sensors, which may be arranged in a grid pattern. Referring to FIG. 6, a foot 60 is shown, illustrating multiple regions 61, 62 that may be illuminated in some aspects of the invention. At least one heel location 61 may be illuminated, along with at least one location 62 nearer to the toes.

In an alternative embodiment the apparatus may comprise part of an insole 70 or shoe 80 (as shown in FIG. 8. Where the apparatus is part of a shoe the housing 10 may for instance be within the sole of the shoe 80 (e.g. the heel). Such an arrangement may allow the light source 22 to directly illuminate the illuminated region of a foot within the shoe, with the need for a coupling means (such as an optical fibre). Similarly, the light detector 24 may be arranged to directly receive the light from the illuminated region. Alternatively, the light source 22 and light detector 24 may coupled to the illuminated region 36 by a light guide, such as an optical fibre 2. An embodiment that is part of a shoe 80 may require the use of socks with a transparent region, or wearing of the shoe 80 without socks, so as to allow the foot to be illuminated.

An apparatus comprised as part of an insole 70 may be inserted into a user's shoe, allowing them to continue to wear their normal shoes while the circulation in their feet is monitored. A low profile battery may be integrated with the insole 70, and the electronics from the housing 10 in the embodiment may be embedded within a foam material. The electronics may be formed on a flexible printed circuit board.

In some embodiments the apparatus is not wearable. In such device the support element 14 may comprise part of a platform 50 on which the user may stand, as shown in FIG. 5. The platform 50 may comprise a plurality of support elements 14 for receiving the weight of the user. Each support element 14 may comprise at least one light source 22 and light detector 24, for illuminating at least one region of the user's foot on the support element 14. In such an arrangement the apparatus may be operable to determine a capillary refill rate when a user shifts their weight from one foot to the other without lifting their feet. A display 52 may be provided for displaying results derived from the capillary refill test, either in addition or as an alternative to transmitting data related to the capillary refill test. Pressure and temperature sensor 16s may be provided at each support element 14.

In some embodiments the apparatus is a weighing scale, and the display 52 is operable to indicate the weight of a user standing on the platform 50. The display 52 may be operable to provide instructions to the user in relation to performing a capillary refill test.

The scale may be configured to schedule circulation checks, so that checking of foot circulation becomes integrated with routine weight checking. A scheduled circulation check may be triggered by standing on the scale when a predetermine time has elapsed since the last circulation check. The display 52 may display instructions in relation to performing the circulation check, for example instructing the user how and when to shift their weight.

A method of determining capillary refill rate, and of improving health outcomes, in accordance with an embodiment of the invention, is shown in FIG. 7. The method comprises putting on a wearable device at step 71. In some embodiments this step may instead involve standing on a platform. A region of the user's foot it illuminated in step 72. Step 73 comprises and shifting weight on the support element. In step 74, the light from the illuminated region of the foot is measured during and after weight shift. In step 75 the capillary refill time is determined (e.g. by a processor), based on the measured light. In optional step 76 the capillary refill time may be transmitted (e.g. via Bluetooth® to a computer or smartphone). In optional step 77, a recommendation may be made for the user to change their behaviour based on the determined capillary refill rate. For example, the user's mobile phone (or other personal electronic device) may provide a notification that the user should take the weight off their feet, limit the amount of further walking/running they do, contact a health professional (such as a nurse or doctor), or provide some other message. The message need not be words: it could be an audio or visual or haptic signal.

These embodiments are non-limiting examples, and the scope of the invention is defined by the appended claims.

1. A capillary refill measurement apparatus, comprising
a support element configured to receive the weight of a user through their foot,
a light source for illuminating a region of the foot that exerts weight onto the support element,
a light detector arranged to receive light from the illuminated region of the foot and generate an output based on the received light, and
a processor configured to determine a capillary refill rate from the output of the light detector when the user adjusts the amount of weight received by the support element.

2. The apparatus of claim 1, further comprising a force or pressure transducer, configured to provide an output based on the amount of weight received by the support element.

3. The apparatus of claim 2, wherein the pressure transducer comprises an electrically conductive woven or knitted fabric that changes impedance in response to pressure.

4. The apparatus of claim 1, further comprising a temperature sensor configured to provide an output related to the temperature of the foot, when the apparatus is in use.

5. The apparatus of claim 1, wherein the processor is configured to be responsive to at least one of a pressure on the support element and the temperature of the foot, when determining a capillary refill rate.

6. The apparatus of claim 1, comprising an illumination optical fibre for directing light from the light source to the illuminated region and a sensing optical fibre for directing light from the illuminated region to the light detector.

7. The apparatus of claim 6, wherein the illumination optical fibre and the sensing optical fibre each comprise a stripped portion in which light is transmissible through a sidewall of the optical fibre.

8. The apparatus of claim 7, wherein the apparatus comprises a first and second sensing optical fibre, with a stripped portion of each of the first and second sensing optical fibre positioned on either side of a stripped portion of the illumination optical fibre.

9. The apparatus of claim 6, wherein a tip of the illumination optical fibre that is adjacent to the illuminated region is at an angle of between 25° and 155° to a tip of the sensing optical fibre that is adjacent to the illuminated region, and preferably wherein the tip of the illumination optical fibre is at an angle of between 75° and 180° to the tip of the sensing optical fibre.

10. The apparatus of claim 1, wherein the support element comprises a woven fabric.

11. The apparatus of claim 10, further comprising an illumination optical fibre for directing light from the fight source to the illuminated region and a sensing optical fibre for directing fight from the illuminated region to the light detector, wherein at least one of the illumination and sensing optical fibre are woven or knitted into the fabric of the support element.

12. (canceled)

13. The apparatus of claim 1, comprising a wireless transmitter for transmitting information from the processor to a remote computer.

14. (canceled)

15. (canceled)

16. (canceled)

17. (canceled)

18. (canceled)

19. The apparatus of claim 1, comprising a housing in which the light source, light detector and processor are disposed, the housing comprising connector for removably coupling at least one of the illumination and optical fibre to the light source and the sensing optical fibre to the light detector, and a strap for mounting the housing on an ankle of the user.

20. (canceled)

21. (canceled)

22. The apparatus of claim 1, wherein the apparatus is wearable, for example wherein the support element comprises part of: a sock, and insole and/or part of a shoe.

23. (canceled)

24. (canceled)

25. The apparatus of claim 1, wherein the support element comprises part of a platform for a user to stand on.

26. A weighing scale comprising the apparatus of claim 1, the weighing scale comprising a platform for a user to stand on, wherein the platform comprises the support element and the light source and the light detector are attached to the platform.

27. A method of determining a capillary refill rate comprising illuminating a region of a foot of a user, measuring light from the illuminated region as the user shifts their weight on the illuminated region, and determining a capillary refill time from the measured light.

28. The method of claim 27, wherein the method is performed using the apparatus claim 1.

29. The method of claim 27, comprising comparing the capillary refill rate with a normal value of a capillary refill rate.

30. The method of claim 27, comprising sending an alert if the capillary refill rate deviates from the normal value.

31. (canceled)
